(19) United States
(12) Patent Application Publication
(10) Pub. No.: US 2006/0172303 A1
(43) Pub. Date: Aug. 3, 2006

(54) Method for the separation of cell fractions

(76) Inventor: Lasse Lehnert, Kiel (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/539,738

(22) PCT Filed: Dec. 15, 2003
(86) PCT No.: PCT/EP03/14234

§ 371 Date: Jun. 20, 2005

(30) Foreign Application Priority Data
Dec. 19, 2002 (DE) 102 59 703.0-41

(51) Int. Cl. C12Q 1/68; C12N 5/08

(52) U.S. Cl. 435/6; 435/366

(57) ABSTRACT

The present invention relates to a method for the separation of cells, in particular for the preparation of samples in tumor diagnostics. In particular, the present invention relates to a method for sample preparation for the detection of tumor cells of solid tumors in the course of the diagnosis for prognosis and stratification of therapy, comprising the destruction of cells that make this diagnosis more difficult or entirely impossible. Furthermore, the present invention relates to a kit for the preparation of samples and for the detection of the presence of the altered cells. Finally, the present invention relates to the use of the method and the kit in the diagnostics of altered cells such as tumor cells, and the performance of a PCR reaction to detect the tumor cells in body fluids and tissues.

The present invention relates to a method for the separation of cells, in particular for the preparation of samples in tumor diagnostics. More exactly, the present invention relates to a method for sample preparation for the detection of tumor cells of solid tumors in the course of diagnostics for prognosis and stratification of therapy, embracing the destruction of cells that make this diagnosis more difficult or entirely impossible. Furthermore, the present invention relates to a kit for the preparation of samples and for the detection of the presence of the altered cells. Finally, the present invention relates to the use of the method and the kit in the diagnostics of altered cells such as tumor cells, and the performance of a PCR reaction to detect the tumor cells in body fluids and tissues.

### PRIOR ART

The separation of different cell populations is a central aspect in the analysis of different cell populations. Very different methods are known in the prior art for the separation of cells and these are mostly based on different physical characteristics, or on differences in the expression of certain molecules.

The detection of the presence of circulating and/or micrometastatic altered cells such as tumor cells, in a mixture of cells of different cell populations enables an early assessment of the prognosis of the patient and a stratification for possible, so-called, adjuvant therapy steps.

To enable meaningful early diagnosis, it must be possible to specifically detect the presence of an extremely low number of altered cells in the sample to be analyzed.

Tumor cells and other altered cells virtually always have genetic alterations compared to their normal precursors. The alterations relate either to the appearance of tumor-associated and characteristic gene expression profiles and/or are characterized by specific gene mutations. The detection of the presence of the latter (for instance mutations in the genes p53, BRCA1 & 2, APC and others) has not so far been technically possible at the DNA level with the necessary diagnostic sensitivity if the tumor cells are under-represented relative to normal cells in the sample.

In contrast, detection of the presence of altered cells, such as tumor cells, at the required sensitivity is possible with the amplification of a so-called “tissue-specific mRNA expression” (molecular staging), provided that the following pre-requirements are met:
a.) The source organ of the tumor is known. Tissue-specific mRNA markers can be defined, such as
   i) Cytokeratins, that represent general epithelial markers, and the presence of which remains detectable for malignant tumors of epithelial origin (carcinomas).
   ii.) So-called differentiation markers of different tissue such as, for example, CEA (for large intestine carcinomas), PSA (for prostate carcinoma), AFP (for hepatic carcinomas), tyrosinase (for malignant melanoma) etc.
b.) The normal cells otherwise still present in the test sample (normal blood leucocytes of a blood sample) do not express the mRNA marker used as a target for molecular staging: conversely, the mRNA markers that are expressed in normal cells are not suitable for staging.

If the pre-requirements are satisfied, then the presence of roaming altered cells, such as tumor cells, can be detected in a test sample via the tissue-specific mRNA expression profile using suitable sensitive methods, in contrast to specific-specific gene mutations, also within a large excess of normal cells.

In technical terms, the detection is based on the in vitro production of a cDNA copy of the target mRNA using reverse transcription, with a subsequent DNA polymerase chain reaction (RT-PCR). This detection is possible with a high sensitivity and generally embraces a single tumor cell in 10⁶-10⁷ normal blood cells (i.e. 1 ml blood).

To date, however, severe restrictions have been placed on the use of the RT-PCR test. A large number of tissue-specific expressed genes have been described that are, in principle, suitable for the detection of the presence of altered cells derived from that tissue, for example tumor cells. The detection of their presence, however, is not always successful for the following reasons, as a result of system-immanent special characteristics of such amplification systems, given below:

The analytical and diagnostic sensitivity of detection methods based on RT-PCR is very high. In other words, the method enables the reliable detection of individual molecules in test systems and in clinical test material. In addition, the analytical specificity is very high. In other words, any erroneous amplification of molecules, other than the desired mRNA molecules, through so-called cross-hybridization of the primer, can be ruled out with certainty under suitable conditions.

However, the diagnostic specificity is insufficient. This means that positive results are regularly found for the selected “tumor-associated” target mRNA in clinical samples from normal individuals or patients with non-malignant disease. Non-malignant cells in the sample can therefore give rise to positive RT-PCR results. Correspondingly, it is difficult to interpret the results in individual cases for diagnosis purposes.

The resultant low diagnostic specificity is the reason why the detection of the presence of circulating tumor cells through RT-PCR has not been widely adopted for the diagnosis of minimal residual disease (Jung et al., EJCCCB, 1997, Jung et al., J Lab Med. 1999). The false-positive results, in fact, do not permit the use of this intrinsically very advantageous method.

The main reason for the insufficient diagnostic specificity is mRNA background expression (also referred to as illegitimate transcription or background transcription) due to the presence of normal cells in the test sample (for example, white blood cells in blood and bone marrow). It has been shown that normal white blood cells can express tumor-associated mRNA markers. This “illegitimate” expression has a low value at the level of the individual cell, but results in a marked measurable signal because of the high number of these naturally-occurring cells in the sample, and thus leads to the aforementioned non-specific positive results. The pattern of background expression may be constitutive (Jung et al., Br. J. Cancer (1999)), or induced, for instance in the course of an inflammation reaction (Jung et al., Br. J. Cancer, (1998)).

It is evident from the literature since then that the problem of background expression substantially reduces the utility of the method. Correspondingly, various methods have been proposed to increase specificity and exploit the physical or expression-specific characteristics of the cells to be separated, similarly to the usual separation methods. These methods, however, frequently have substantial diagnostic restrictions for their clinical utility.

The most commonly used method for separation of cell populations, for instance from whole blood, is the so-called FICOLL density gradient centrifugation. A large number of variants of the same principle exist for FICOLL gradients. This is based on an enrichment of mononuclear cells in which the presence of a tumor cell is then detected.

The FICOLL method cannot be standardized, i.e., it is of varying effectiveness in the recovery of separated cells. A main determinant of the sensitivity of the detection of the presence of tumor cells is the technical expertise of the investigator (Krüger et al., Clin. Chem., 2000). In addition, the recovery and enrichment of altered cells, such as tumor cells, is uncertain because of their unpredictable sedimentation behavior. In summary, it is unclear whether tumor cells are lost in the course of sedimentation, and if so how many, and whether the tumor cells in general behave sufficiently homogenously in terms of their specific density that they can always be separated together with mononuclear cells and are not sedimented in the discarded granulocyte fraction. Recovery rates of between 10 and 70% are reported overall.

The second main principle of the enrichment of target cells is immunobead enrichment. This method is based on the fact that altered cells, such as tumor cells, can be selectively derived from a cell mixture through binding to paramagnetic particles. A pre-requirement for this is the expression of a tumor-associated marker at a suitable density on the surface of the tumor cell. Paramagnetic particles, onto which an antibody directed against this marker has been bound, are adsorbed onto the target cells and are then enriched from the solution through the use of a magnet. The successful binding of the antibody requires a sufficient density of the marker on the cell surface.

The immunobead enrichment method has two limiting pre-requirements:

1) The marker has to be a surface marker and is therefore dependent on the tumor type. However, the heterogeneity of carcinomas is such that there is no uniformly high and suitable expression of the marker on all individual tumors or their metastases. The number of molecules on the surface required for successful enrichment, is essentially unknown.

2) To allow treatment of the cells in an intact state, the method requires the treatment of native test samples directly following the sampling procedure. A degradation of the sensitive mRNA has been observed within a period of hours after blood sampling (Gerhard et al., J. Clin. Oncol. 1994 Apr; 12 (4): 725-9). The associated decrease in mRNA quality rapidly reduces the diagnostic sensitivity of a RT-PCR test. It follows that both the sample preparation and the time factor for the immunobead separation are of decisive importance for the detection of the presence of individual tumor cells on the basis of tissue-specific mRNA. These pre-requirements are not met for routine diagnostic use.

The U.S. application Ser. No. 2002/0012931 describes a method for the detection of the presence of circulating tumor cells, in which the cells responsible for false-positive signals in the detection of GC-C (guanylyl cyclase C) were identified as CD34-positive cells in the blood. According to the method described therein, these false-positive cells should be removed from the cell mixture through elimination, for instance through immunobeads. The presence of the GC-C mRNA in the remaining cells is demonstrated thereafter. This method therefore involves a negative selection of the altered cells, i.e., the cells responsible for the false-positive signal are separated out.

An imperative requirement for both of the methods outlined above is that the sample be treated further immediately after sampling to ensure that the integrity of the mRNA within the derived cells is maintained.

The aim of the present invention is to remove these restrictions, in the standardized performance of the method as well. This applies, in particular, to those cases where RT-PCR is to be used in the course of routine diagnostic procedures after the separation procedure. The method according to the invention thereby has to satisfy the following pre-requirements:

1) It must be possible to easily remove the cells (sub-populations) in the test sample responsible for illegitimate expression.

2) The above-mentioned sub-populations to be separated and their constituents should be “removed” from the sample. The mRNA expression by the background cells must be overcome through this removal.

3) It must be possible to perform the method with a minimum of effort at the place of sampling in a reliable manner so that the method is standardized. Standardized in this context means firstly that the systems used for sampling must lend themselves to pre-fabrication. On the other hand, it is necessary for the sampling protocol to be devised such that the method can be performed by different investigators with equal efficiency when proceeding in the same manner. By definition, it is not possible to use a technically non-standardized method (such as the FICOLL density gradient or immunoseparation) for this purpose.

4) The method must not be linked to the necessity of maintaining complex technical apparatus at the place of sampling, as it would then no longer be suitable for broad usage on a routine basis.

5) It must be possible to perform the method rapidly enough to avoid endangering the integrity of the mRNA in the sample. If this is not the case then the diagnostic sensitivity would be critically reduced.

### BRIEF DESCRIPTION OF THE INVENTION

To satisfy the above requirements, the method according to the invention for separation of normal cellular sub-population(s)/fraction(s)—as defined below—from the altered cells in a sample embraces the step of incubating the mixture of normal cells and altered cells in a hypotonic solution and destruction of fractions thereof.

The method is based on a novel concept of destroying one or more different cellular sub-populations/fraction(s) responsible for the illegitimate expression of the mRNA marker under investigation in the solution through hypotonic influence before the sample is processed further for analysis of the altered cells, in contrast to the methodology commonly adopted to date.

The method according to the invention can, furthermore, embrace the step of collecting and recovering the non-eliminated cells. If the method is to be used for further analysis of the collected cells, then an analysis step, for example a RT-PCR, is used after the recovery step.

It was found that incubation in a hypotonic solution results in a differentiated destruction of cell fractions in the samples. In particular, altered cells, such as tumor cells, have a greater resistance to the hypotonic conditions in the solution than cell populations normally present in the sample.

The method according to the invention can be used to detect the presence of altered cells, such as tumor cells. One possibility is the use of the method for the diagnosis of metastasizing cancer.

The invention provides for a kit for the detection of the presence of altered cells, such as tumor cells. This kit embraces a hypotonic solution, or means of rendering the solution hypotonic, and primers for detection of mRNA coding for a specific-specific marker. Furthermore, the kit preferably embraces a RNA-stabilizing solution, comprising a highly-concentrated chaotropic salt.

This kit can be used to diagnose metastatic cancer.

### BRIEF DESCRIPTION OF THE FIGURES

FIG. 1: FIG. 1a shows the detection of the presence of cytokeratin 20 (CK20) and (PBGD) under different hypotonic conditions for normal blood. FIG. 1b shows the detection of the presence of cytokeratin 20 (CK20) and (PBGD) under different hypotonic conditions for normal blood with a certain number of tumor cells.

FIG. 2: FIG. 2 shows the detection of the presence of cytokeratin 20 (CK20) and (PBGD) under different hypotonic conditions for normal blood with 25 tumor cells (HT29).

FIG. 3: FIG. 3 shows the relative proportion of the granulocyte sub-population within the blood cells as a function of the treatment with hypotonic solutions.

### DETAILED DESCRIPTION OF THE INVENTION

The following terms, as used herein, are explained in more detail, to assist in understanding the application.

Osmolarity/osmolality: Osmolarity is the concentration of osmotically-effective particles in 1 liter of test material (osm/L). Osmolality is the concentration of osmotically-effective particles in 1 kg solvent (osm/kg). Milliosmoles are abbreviated to mosm.

Hypotonicity: Solutions of the same osmotic pressure are isotonic. Solutions of increased osmolality are termed hypertonic and solutions with a lower osmolality as hypotonic.

Destruction of cells: The removal of cell integrity through chemical/biological or physical methods. An example is the addition of a hypotonic liquid to cells to burst them.

Tumor-associated/specific mRNA: In the course of gene expression one or more “copies” of a gene (DNA) is produced in the form of mRNA (messenger RNA).

This process is referred to as transcription. Thereafter, the genetic information of the mRNA is converted to an amino acid sequence (known as translation). 3o This results in proteins that can assume multiple roles. Tumor-specific/associated RNA molecules are characteristic for tumors and are either not formed by normal cells, or frequently in small quantities. The mRNA coding for a tumor-associated molecule is thus the precursor of the tumor marker detectable by immunocytochemical methods.

Solid tumors: Solid tumors are distinguished from non-solid tumors through the formation of a measurable, coherent tumor mass. Carcinomas are an example of a solid tumor and leukemia is an example of a non-solid tumor.

Stabilizing: In the context of the invention, stabilization is firstly a prevention of the degradation of RNA and, secondly, the protection of tumor cells against destruction by a hypotonic solution.

Cell constituents: Cell constituents comprise all structures, substances and molecules that define the cell in its form and function. These include, for example, the cell nucleus, the cell membrane, the cytoplasm, DNA, RNA and proteins etc.

Cells: Cells in this context are all cells, in their entirety, that make up a human being.

Cell types: The types of cells are, for example, blood cells or tumor cells, intestinal epithelial cells etc.

Cell populations: These are sub-groups within a cell type, such as white blood cells (leukocytes) and red blood cells (erythrocytes) within the blood cells.

Sub-populations: These are sub-groups within the cell populations, such as, for example, granulocytes and lymphocytes within the white blood cells (leukocytes).

Cell fractions: Cell fractions are sub-groups within the sub-populations, for example eosinophils and basophils within the granulocytes.

Normal cells/non-tumor cells: These are all of the cells that are not malignant or altered in any other way. In the present case, normal blood cells are assumed. Altered cells, such as tumor cells or precursors of tumor cells, exist in contrast to these.

Background expression: Altered cells, such as tumor cells, express large quantities of certain marker molecules (for example tumor-associated mRNAs such as CK20 mRNA, CEA mRNA or PSA mRNA) that are characteristic for certain cell types such as epithelial cells. Some normal blood cells express very low quantities of these mRNAs. The presence of a large number of normal blood cells in a sample will therefore generate a signal that corresponds to that of a low number of tumor cells producing the marker molecule in large quantities.

This expression of marker molecules at a very low level by normal blood cells is defined herein as background expression. Synonyms for background expression are illegitimate transcription and background transcription

CP value: The “Crossing Point” (CP value; threshold cycle) of a PCR reaction measured in a LightCycler® (Roche) is the PCR cycle at which PCR amplification enters the exponential phase. What is meant is the exact time point of the PCR reaction (cycle number) at which the fluorescence of a certain reaction exceeds the background fluorescence. This time point is most reliably proportional to the concentration of the template present at the start of the amplification process. This time point is automatically established by the Roche LightCycler software in graphical form.

The lower a CP value, the more copies of the nucleic acid section to be amplified are present in the sample. If no CP value is established, then a sample does not contain a target molecule that can be amplified.

Porphobilinogen Deaminase (PBGD): PBGD is a so-called “housekeeping gene” and is expressed constitutively at a low level by all somatic cells. It acts as a positive control for the presence of mRNA in the sample.

The invention provides a method for the separation of cell fractions comprising normal cells and altered cells. The cell fractions comprising normal cells and altered cells are incubated in a hypotonic solution. One or more cell fractions are destroyed in this process.

The invention is based on the finding that cells differ in their resistance to a hypotonic solution before cell integrity is destroyed. More exactly, it was found by the inventors that altered cells, such as tumor cells, are more resistant to hypotonic influences than normal cells.

This new property of altered cells relative to normal cells 2 incubation in a hypotonic solution.

The step of incubation in a hypotonic solution may be followed by a purification step. The non-disintegrated cells are collected in this step. The cells thus obtained can then be subjected to analysis. This analysis can, for example, take the form of analysis using RT-PCR.

The analysis of the derived cells can embrace the determination of the expression of a tumor marker, such as associated/specific mRNA. In particular, when this method forms part of a diagnostic method for tumors, such as circulating and micro-metastatic tumor cells.

The method according to the invention enables the separation of cell fractions, comprising cells that are responsible for the background expression of a tumor marker, from altered cells, such as tumor cells, that may be present in the test sample at a low number. The method according to the invention allows the detection of the presence of altered cells, such as tumor cells, in test samples that would not normally be possible because of the background expression of the selected tumor marker by normal cells. The background expression through normal cells can lead to a false-positive signal in the test sample.

The test samples may be mixtures of normal cells and altered cells from body fluids or tissue. In particular, the body fluid may be one of the following: Blood, urine, cerebrospinal fluid, bone marrow, lymph, ascites or sputum.

As already mentioned above, the altered cells may be tumor cells. Preferred are tumor cells, circulating and/or micro-metastatic tumor cells of solid tumors such as carcinomas in tissues and body fluids, where this type of tumor does not occur.

In particular, the micrometastatic tumor cells are tumor cells of epithelial origin.

In a preferred embodiment of the invention the test sample is bone marrow or whole blood.

The normal cells of the test sample, which can express one or more tumor-associated mRNA species are:
Cells of the myelotic or lymphatic differentiation series at different stages of maturity. These include undifferentiated myeloblasts and stages of maturity through to segmented granulocytes and monocytes/macrophages; undifferentiated megakaryoblasts and maturity stages through to thrombocytes; proerythroblasts and maturity stages through to reticulocytes.

The lymphatic series includes leucocytes of the Iymphatic series at different stages of differentiation, in particular lymphatic stem cells and maturity stages through to differentiated effector cells of the T-lymphocyte/B-lymphocyte series.

According to the invention these normal cells, or non-tumor cells, that express the associated-associated mRNA either in a quiescent state, or only in an activated/stimulated state, are eliminated from the sample only upon use of a hypotonic solution before the detection of the presence of tumor cells.

The hypotonic solution has an osmolality below 100 mosm/kg. The preferred osmolality of the solution is in the range 30-60 mosm/kg, more exactly 40 mosm/kg.

The hypotonic solution can be added as such to the cells or with the use of auxiliary agents such as Sephadex, active charcoal or ion exchangers to lower the osmolality of the solution. A preferred hypotonic solution is based on a solution of salts such as, for example: NaCl, KCl, NH₃Cl, phosphate buffered saline (PBS), Hank's Balanced Salt Solution (HBBS) and mixtures thereof. Alternatively, pure water may be used.

The hypotonic solution can contain further adjuvants that promote the disintegrating effect of the hypotonic solution or accelerate the degradation of constituents of the disintegrating cells. These adjuvants can be ionic and non-ionic tensides such as, for example, saponin, Triton, Tween, sodium dodecyl sulfate (SDS). Further adjuvants are enzymes that degrade nucleic acid (RNases and DNases) and/or protein-grading enzymes (proteinase K, pronase or others).

In a preferred embodiment, the hypotonic solution contains enzymes that degrade nucleic acid and/or protein-degrading enzymes, such as RNase.

In view of the general instability of cellular mRNA, it is advantageous for the elimination of non-tumor cells to be carried out rapidly and directly after sampling and at the same location. The short lapse of time ensures that the integrity of the test sample is maintained and also enables the presence of small quantities of tumor cells in the sample to be detected (diagnostic sensitivity).

According to the invention, the elimination (destruction) is carried out through incubation of cells of the test sample in a hypotonic solution. The constituents of the cells, such as RNases, released in the process, or artificially-added constituents, such as enzymes, including RNases, can degrade the damaged or lysed cells, and thus, in particular, remove their mRNA - and thereby the illegitimate mRNA transcription products.

The invention provides for a subsequent so-called stabilization of the sample. This stabilization step can be performed before or after recovery of the non-destroyed cells. If stabilization is carried out after the recovery of the non-destroyed cells then the solution will result in the lysis of all remaining cells, including the altered cells, in particular tumor cells, with concurrent stabilization of the mRNA of those cells. With the lysis it must be ensured that free enzymes that exhibit a nucleic acid-degrading activity, are deactivated so that they do not destroy the associated-associated mRNA of the altered cells, such as tumor cells, released at the same time.

This solution can contain a highly-concentrated chaotropic salt (for example guanidinium isothiocyanate or guanidinium hydrochloride) for stabilization of the RNA.

The tumor-associated/specific mRNA, the presence of which is demonstrated in the analysis of the derived cells, may be selected from cytokeratin 18 (CK18), cytokeratin 19 (CK1 9) and cytokeratin 20 (CK20), as well as other members of the cytokeratin family, carcinoembryonic antigen (CEA), ErbB2, ErbB3, epithelial mucin-1, epithelial mucin-18, guanylyl cyclase C, Cdx-1, Cdx-2, prostate specific antigen (PSA), prostate specific membrane antigen (PSMA), sucrose isomaltase, lactase, carbonic anhydrase, tyrosinase, thyroglobulin, tyrosine hydroxylase, neurone-specific glycoprotein, desmoplakin l, epithelial glycoprotein 40 or gastrointestinal tumor-associated antigen.

A potentiation of the lysis effect of the hypotonic solution on cell fractions of blood is possible through the use of specific antibodies against the cell fractions to be destroyed through hypotonic lysis, coupled with the use of complement. Cells, for example, the granulocytes, are pre-damaged in this process by antibodies directed against surface antigens, e.g. CD123, CD125 and complement in a way that a less hypotonic solution (>100 mosm/kg) is sufficient in a following step to lyse the target cells. A pre-requirement is that the antibodies used are capable of triggering complement lysis.

Antibodies that are not able to mediate complement lysis can, however, similarly be used in an embodiment form according to the invention to supplement the system. Such antibodies are directed against surface antigens on the tumor cells, to which they bind, and stabilize them, so that the tumor cells to be detected also remain intact at a very low osmolality (<15 mosm/kg).

It is clear that the method according to the invention can be used in combination with known methods.

The kit according to the invention for the detection of tumor cells in a sample comprises a hypotonic solution and primer for detecting the presence of mRNA coding for a marker for altered cells, such as tumor cells. This marker can take the form of a tumor-associated/specific mRNA. If the analysis embraces the detection of mRNA, the kit advantageously contains a RNA-stabilizing solution, comprising a highly-concentrated chaotropic salt.

The hypotonic solution, or means to bring about hypotonic conditions, s contained in the kit results in an osmolality below 100 mosm/kg. The preferred osmolality range of the hypotonic solution is 30-60 mosm/kg.

The kit according to the invention can, in particular, be used under routine diagnostic conditions for the diagnosis of metastatic cancer. The presence of tumor cells in the test sample is detected without a false-positive signal resulting from the illegitimate expression of the marker by normal cells.

The invention is described below in more detail through examples. These examples, however, are in no way limiting for the invention.

### EXAMPLE

### 1. Production and Measurement of Defined Hypotonic Solutions

Defined hypotonic solutions:

Osmolality was determined using the following apparatus: Fiske Osmometer, model 2400 Multisample (Dr. Berthold G. Schlag Wissenschaftliche Messinstrumente Nachf. GmbH, Am Muhlenberg 19, D-51465 Bergisch Gladbach, Germany). This is a commonly-used instrument for determination of osmotic pressure through depression of the freezing point. The instructions of the manufacturer were followed.

Serial dilutions of PBS (Phosphate Buffered Saline, BioWhittaker, BE17-516F, 0.0067 M (PO₄)) were prepared. Undiluted solution was assigned a value of 100%. Various hypotonic solutions were produced and their osmolality determined (P-solutions), see Table 1.

TABLE 1

| % PBS | Osmolality (mosm/kg) |
|---|---|
| P100 | 287 |
| P35 | 103 |
| P30 | 84 |
| P25 | 72 |
| P20 | 58 |
| P15 | 42 |
| P10 | 26 |
| P5 | 13 |
| P1 | 2 |

Serial dilutions of HBSS (Hank's Balanced Salt Solution, (PAA, Cat No. H15-012) were prepared. Undiluted solution was assigned a value of 100%. Various hypotonic solutions were produced and their osmolality determined (H-solutions), see Table 2.

TABLE 2

| % HBSS | Osmolality (mosm/kg) |
|---|---|
| H100 | 276 |
| H50 | 147 |
| H35 | 101 |
| H30 | 86 |
| H20 | 56 |
| H15 | 46 |
| H10 | 24 |
| H5 | 11 |
| H1 | 0 |

The osmolality of the solutions was determined as follows:

Sample volume: A sample volume of 20 μl, calibrated for measurement, was introduced into the measurement cell of the instrument. Measurement was carried out using appropriate internal quality controls to ensure good precision of the measurements.

Procedure:

20 μl of each different dilution of an isotonic starting solution was pipetted in its entirety, without bubble formation, onto the base of the sample vessel in a sample ring. Measurement was carried out fully automatically for up to 20 samples and the measured values printed out through the integrated printer.

### Reference Example

A) Blood cells alone:

Blood from volunteer test subjects was used. Coagulation was prevented by adding lithium-heparin in advance.

1. 2 ml heparin-Li whole blood was distributed over the reaction vessels (with 18 ml of the corresponding H/P solution introduced into each reaction vessel in advance). The solutions were mixed through rotation and incubated at room temperature for 15 minutes.

2. The cells were centrifuged at 350× g, the supernatant discarded and the pellet was resuspended in 10 ml lysis buffer (R&D Systems, Cat No. WL1000). Mixing was performed by means of shaking, not vortexing.

The first step ensures the elimination of the cells responsible for the “false-positive” background through hypotonic shock. In the second step the remaining cells (altered cells/tumor cells) are recovered.

Commercial standardized systems are available for isolation of RNA from this step onwards and are mainly based on the method of Chomczynski and Sacchi (Anal Biochem. 1987 Apr; 162 (1): 156-9). In the case described here, the RNA was isolated using a kit from the company Qiagen (QlAmp RNA Blood Mini Kit, Cat. No. 523003, Qiagen, Hilden). The instructions of the manufacturer were followed.

3. The isolated RNA was eluted from the membrane with 30μl ddH₂0 and concentrated in a vacuum centrifuge. The pellet was then resuspended in 13 μl ddH₂O.

4. 3 μl was used for determination of the RNA concentration.

5. 10 μl RNA was used for the cDNA synthesis. cDNA synthesis is a constituent part of the Roche CK20 PCR kit (Cat. No. 3118 835) and was performed in accordance with the instructions of the manufacturer.

6. 4μl of the cDNA obtained was introduced into the CK-20 LightCycler PCR and the instrument used in accordance with the instructions of the manufacturer.

7. The studies were evaluated using LightCycler Software 3.5.

B) Blood cells with admixed tumor cells:

1. HT29 colon carcinoma cells were separated from their substrate using Accutase™ (PAA-Laboratories GmbH, Cat No. L11-007) at a volume of 3m1 per T175 culture flask. Cell culture medium (RPMI 1640+10% FCS+2mM glutamine+1 mM sodium pyruvate, Invitrogen, Life Technologies Karlsruhe) was then added to make the volume up to 30 ml to inactivate the Accutase and the cells were centrifuged (1400 rpm for 5 min.). The pellet was resuspended in 20 ml and the cell count was determined using a Neubauer cell counting chamber. The desired cell number per ml was then derived by further dilution with culture medium.

If >100 cells per run were added per aliquot, then the cell number was adjusted through dilution. For cell numbers <100, the tumor cells were counted out of the resuspended tumor cell suspension using a micromanipulator and transferred to a reaction vessel in which 100 μl PBS or HBSS was introduced in advance. The cells were admixed with the blood through the addition of 100 μl prior to the blood (2ml) with the corresponding P/H solutions.

The further steps in the procedure and the evaluation were as described under A) 1-7.

### Example 1

Blood was taken from a blood donor and divided into 2 aliquots. The first aliquot was treated on its own and for the second aliquot after addition of 1000 HT29 tumor cells. Since only 20% of the cDNA generated can be used for the CK-20 PCR, the signals correspond to those for 200 HT29 cells. The samples were treated and analyzed in the same manner as the reference example.

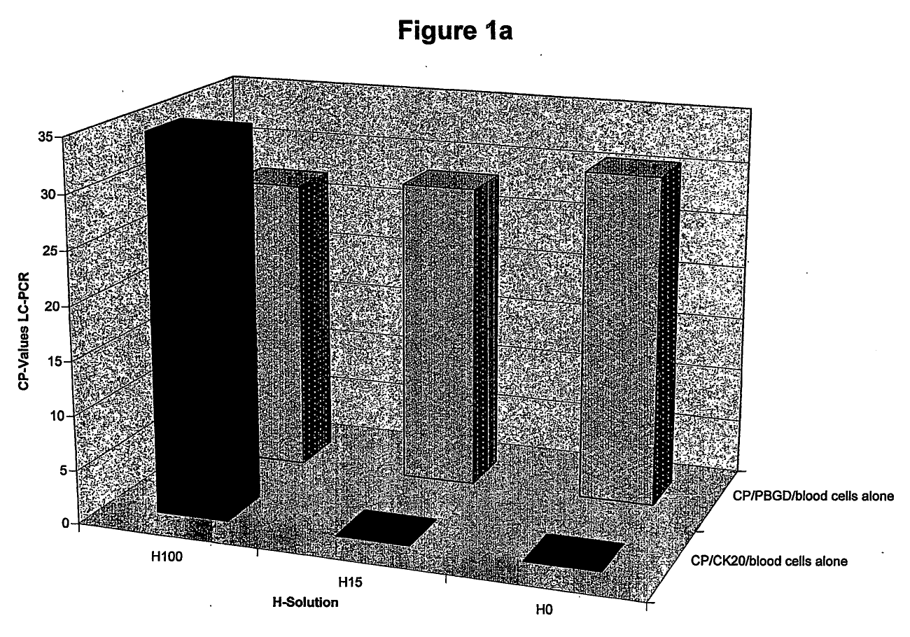

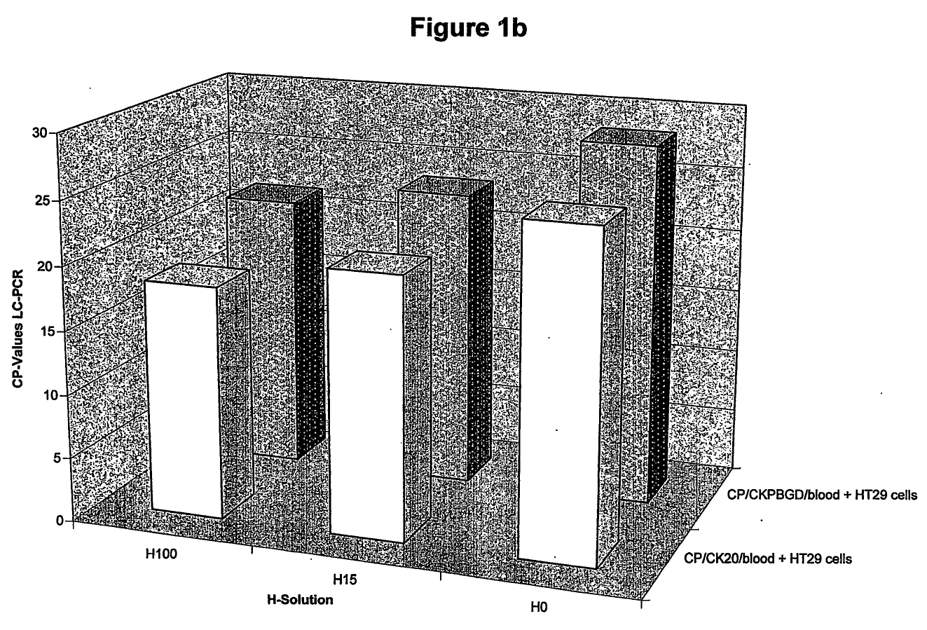

FIG. 1a shows the results for the test run without addition of tumor cells. CK20 and PBGD signals are detected for the blood cells treated with a solution of 276 mosmoles (H100). No CK20 signal is seen after treatment with H solutions (H15-HO) with a lower osmolality (<46 mosm/kg), but the PBGD signal is present (sufficient sample material was therefore present).

FIG. 1b shows the results for the test run with tumor cells added (HT29). CK20 and PBGD signals are also detected after treatment of the sample with solutions of low and very low osmolality (H15, HO). Since no CK20 signal was seen in the parallel test run without addition of tumor cells at an osmolality below 46mosm/kg, these signals are clearly attributable to the added tumor cells.

The results shown in the Figures are representative extracts from several test runs with different hypotonic solutions. The results of experiments that go beyond the test runs shown in the Figures are summarized in Tables 3 and 4.

TABLE 3

| | Positive detection of the | Positive detection of the |
|---|---|---|
| | CK20 signal | PBGD signal |
| | (Blood cells alone) | (Blood cells alone |
| | No. of positive detections/ | No. of positive detections/ |
| H solution | No. of test runs | No. of test runs |
| H100 | 6/6 | 6/6 |
| H20 | 2/6 | 6/6 |
| H15 | 0/8 | 8/8 |
| H10 | 0/4 | 4/4 |
| H5 | 0/3 | 3/3 |
| H1 | 0/3 | 3/3 |
| H0 | 0/4 | 4/4 |

Table 3: Detection of the CK20/PBGD signal in blood cells as a function of the hypotonic salt solutions used (H solutions).

The use of a 56-mosmolar solution (H20) led to a 60% elimination of the CK20 signal, with 100% detection of the PBGD signal. A 100% elimination of the CK20 background signal was achieved with a solution of approximately 42 mosmoles (H1 5). The PBGD signal was also 100% retained.

TABLE 4

| | Positive detection of | Positive detection of |
|---|---|---|
| | the CK20 signal | the PGBD signal |
| | (Blood cells + HT29 | (Blood cells + HT29 |
| | tumor cells) | tumor cells) |
| | No. of positive detections/ | No. of positive detections/ |
| H solution | No. of test runs | No. of test runs |
| H100 | 3/3 | 3/3 |
| H20 | 3/3 | 3/3 |
| H15 | 3/3 | 3/3 |
| H0 | 3/3 | 3/3 |

Table 4: Detection of the CK20/PBGD signal in blood cells with added tumor cells as a function of the hypotonic salt solutions used (H solutions).

Both the CK20 and the PGBD signal were seen under all test conditions. This means that tumor cells are resistant to the hypotonic solutions used and thus can be differentiated from the blood cells.

Further studies, in which a low number of tumor cells were added, showed that CK20 signals could be detected for 5 tumor cells.

### Example 2

Blood was taken from a blood donor and split into 2 aliquots. The first aliquot was used without addition of tumor cells the second aliquot was mixed with 25 HT29 tumor cells.

The samples were prepared and analyzed in the same manner as the reference example.

Since only 20% of the cDNA generated can be used for the CK-20 PCR, the s signals correspond to those for 5 HT29 cells. For this donor, the background signal was reached with hypotonic solutions at an osmolality of just 103 mosm/kg.

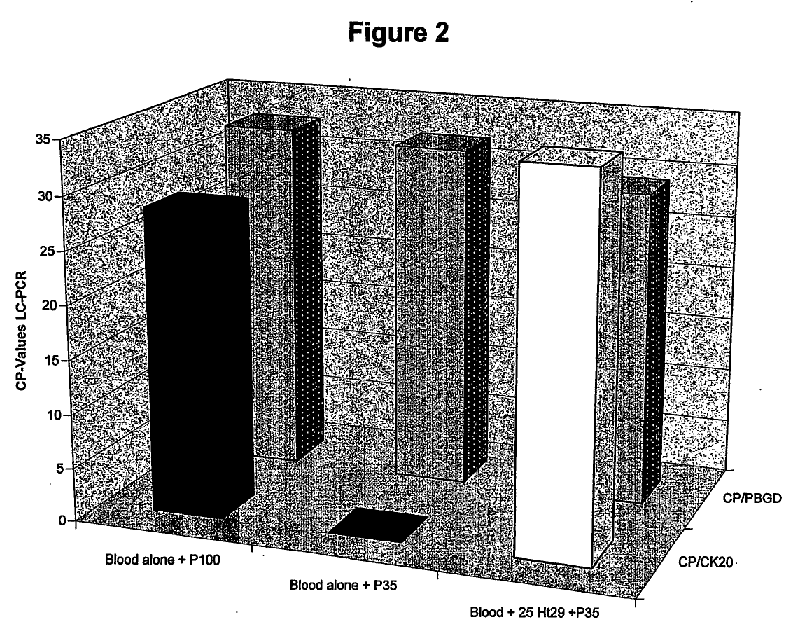

FIG. 2 shows the results of quantitative RT-PCR for CK20 and PBGD. The CK20 and PBGD signals can be detected without treatment of the blood cells with hypotonic solutions (blood alone). Incubation with a P35 solution (103 mosm/kg) leads to elimination of the CK20 signal. The PBGD signal is retained (with P35). In the parallel run, containing 25 HT29 tumor cells, the CK20 signal remains detectable (blood+25Ht29+P35).

### Example 3

### Improvement of the Results of Lysis Through Addition of RNase A

Example 1 was repeated. RNase A (1 mg/aliquot) was added to the blood before treatment with the P/H solution to enable rapid degradation of the RNA before the final lysis through the RLT buffer containing guanidinium isothiocyanate. This led to an improvement in the results of lysis, i.e., lysis was also possible with solutions of higher osmolality.

### Example 4

### Primary Effect of P/H Solutions on Granulocytes as a Cell Fraction of Normal Cells in the Test Sample

The finding that the CK20 signal is expressed by the granulocyte fraction of the leukocytes, underlines the results presented here. FACS analysis was carried out to investigate the composition of the blood cell fractions, with and without P/H solutions, and a marked reduction in the number of granulocytes was observed in agreement with the CK20 data.

The most marked reduction was observed at an osmolality of 25-40 mosm/kg.

The test procedure was as follows:
1. 4 ml heparin blood was mixed with 100ul antibody solution directed against the surface antigen CD45/CD14 (Simultest LeucoGate, BD-Biosciences, Cat. No. 342408) and CD16 (Caltag, Cat. No. MHCD1606) in accordance with the instructions of the manufacturer and incubated at room temperature (RT) for 15 minutes.
2. 24 FACS tubes, each containing 2 ml H-solution, were prepared.
3. 150μl blood was added to each 2 ml of H-solution and mixed well, followed by incubation for 15 minutes at room temperature.
4. The tubes were centrifuged for 5 minutes at 300× g.
5. The pellet was resuspended in 2-3 ml erythrocyte lysis buffer (R&D Systems Inc., Cat. No. WL1000).
6. The samples were incubated for 10 minutes at room temperature.
7. The samples were centrifuged for 5 minutes at 300× g.
8. The pellet was washed in 2-3 ml wash buffer (R&D Systems Inc., Cat. No. WL1000).
9. The sample was again centrifuged for 5 minutes at 300× g and washed with 5ml Cellwash (BD Biosciences Cat. No. 349524). It was then re-centrifuged for 5 minutes at 300× g.
10. The pellet was resuspended in 500 μl Cellwash (BD Biosciences Cat. No. 30 349524), and 60 μl fixative (R&D Systems Inc., Cat. No. WL1000) added
11. FACS measurement with immunologically-differentiated surface was performed with a FACSCalibur flow cytometer (BD Biosciences, Germany).

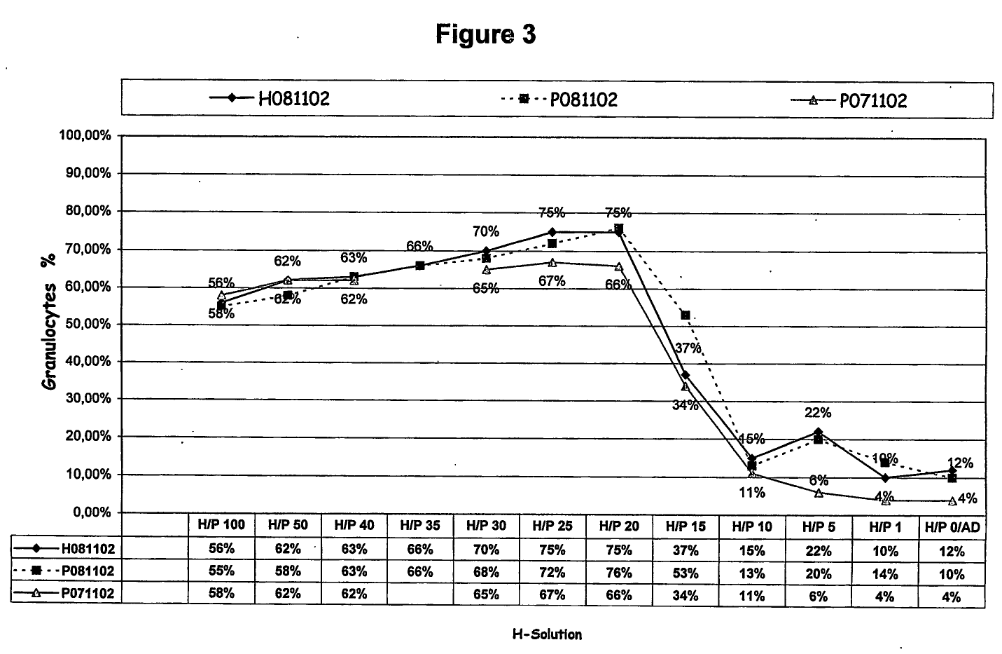

FIG. 3 shows the relative proportion of granulocyte subpopulations of blood cells within the blood cells as a function of treatment with hypotonic solutions. A reduction in the granulocyte population can be seen that is dependent on the treatment with hypotonic solutions. This reduction is very drastic for solutions in the range 20-40 mosm/kg. The percentage of granulocytes relative to the total cell number fell in this range from approximately 66% to approximately 13%. For solutions <20 mosm/kg, the percentage of granulocytes stabilized at a level of approximately 10% of the total cell number.

Three independent experiments are shown, indicated by different symbols.

1. A method for the separation of cell fractions, comprising normal cells and altered cells, comprising the step of incubating the mixture of normal cells and altered cells in a hypotonic solution and the destruction of one or more cell fractions thereof.

2. A method according to claim 1, further comprising the subsequent step of collecting the non-destroyed cell fraction.

3. A method according to claim 1, further comprising the subsequent step of analysis of the cells of the collected cell fractions.

4. A method according to claim 3, wherein the analysis of the derived cells is through polymerase chain reaction (PCR).

5. A method according to claim 1, wherein the mixture of normal cells and altered cells is derived from bodily fluids or tissue.

6. A method according to claim 5, where the bodily fluid is selected from the group comprising blood, urine cerebrospinal fluid, bone marrow, lymph, ascites and sputum.

7. A method according to claim 1, wherein the altered cells are tumor cells.

8. A method according to claim 1, wherein the tumor cells are circulating and/or micrometastatic tumor cells.

9. A method according to claim 1, wherein the normal cells are only mononuclear cells from the blooc.

10. A method according to claim 1, wherein the tumor cells are selected from the group consisting of the group of solid malignant tumors of epithelial origin (carcinomas).

11. A method according to claim 1, wherein the osmolality of the hypotonic solution is below 100 mosm/kg.

12. A method according to claim 1, wherein the hypotonic solution is a salt solution selected from the salts NaCl, Kcl, NH₂Cl, Phosphate Buffered Saline (PBS), Hank's Balanced Salt Solution (HBBS) or mixtures thereof.

13. A method according to claim 1, wherein the hypotonic solution firther contains enzymes that degrade nucleic acid and/or protein-degrading enzymes.

14. A method according to claim 1, wherein the hypotonic solution further contains Rnase.

15. A method according to claim 3, wherein the analysis of the derived cells comprises the determination of the expression of a tumor marker.

16. A method according to claim 15, wherein the tumor marker is selected from cytokeratin 18 (CK18), cytokeratin 19 (CK19), cytokeratin 20 (CK20) and further members of the cytokeratin family, carcinoembryonic antigen (CEA), ErbB2, ErbB3, epithellal mucin-1, epithelial mucin-18, guanylyl cyclase C, Cdx-1, Cdx-2, prostate specific antigen (PSA), prostate specific membrane antigen (PSMA), sucrose isomaltase, lactase, carbonic anhydrase, typrosinase, thyroglubulin, tyrosine hydroxylase, neurone specific glycoprotein, Desmoplakin 1, epithelial glycoprotein 40 and gastrointestinal associated-associate antigen.

17. Use of a method according to claim 1, to detect the presence of altered cells, in particular tumor cells.

18. Use of a method according to claim 1, for the diagnosis of metastatic cancer.

19. Kit to detect the presence of tumor cells in a sample, comprising
a) a hypotonic solution, and
b) primer to detect the presence of MRNA coding for a tumor marker.

20. Kit according to claim 19, further comprising
c) an RNA-stabilizing solution, comprising a highly-concentrated chaotropic salt.

21. Kit according to claim 19, wherein the hypotonic solution has an osmolality below 100 mosm/kg.

22. Kit according to claim 19, wherein the hypotonic solution is a salt solution, selected from the salts NaCl, Kcl, NH₃Cl, Phosphate Buffered Saline (PBS), Hank's Balanced Salt Solution (HBBS) and mixtures thereof.

23. Kit according to claim 19, for the diagnosis of matastatic cancer.

24. Kit according to claim 19, wherein the marker is selected from the group comprising cytokeratin 18 (CK18), cytokeratin 19 (CK19), cytokeratin 20 (CK20) and firther members of the cytokeratin family, carcinoembryonic antigen (CEA), ErbB2, ErbB3, epithelial mucin-1, epithelial mucin- 18, guanylyl cyolase C, Cdx- 1, Cdx-2, prostate specific antigen (PSA), prostate specific membrane antigen (PSMA), sucrose isomaltase, lactase, carbonic anhydrase, tyrosinase, throglubulin, tyrosine hydroxylase, neuron-specific glycoprotein, Desmoplakin 1, epithelial glycoprotein 40 and gastrointestinal associated, associated antigen.

25. Use of a kit according to claim 19, to detect the presence of tumor cells in a sample.
